(19) United States
(12) Patent Application Publication
(10) Pub. No.: US 2023/0052601 A1
(43) Pub. Date: Feb. 16, 2023

(54) HALO ACTIVE AROMATIC SULFONAMIDE PHARMACEUTICAL COMPOSITIONS FOR INTERNAL USE

(71) Applicant: Rem Brands, Inc., Walton, KY (US)

(72) Inventors: David J. Schneider, Union, KY (US); Jonathan Schneider, Walton, KY (US); Joshua D. Schneider, Union, KY (US)

(21) Appl. No.: 17/877,062

(22) Filed: Jul. 29, 2022

Related U.S. Application Data
(60) Provisional application No. 63/227,014, filed on Jul. 29, 2021.

(51) Int. Cl. A61K 31/18; A61P 31/00; A61K 9/00; A61K 9/107; A61K 9/20

(52) U.S. Cl. CPC A61K 31/18; A61P 31/00; A61K 9/0024; A61K 9/107; A61K 9/20

(57) ABSTRACT

Disclosed herein are compositions useful for the treatment and/or prevention of various diseases and conditions, including a variety of infections, via internal administration to a patient. The compositions comprise an effective amount of a halo active aromatic sulfonamide compound having the structure of Formula (I):

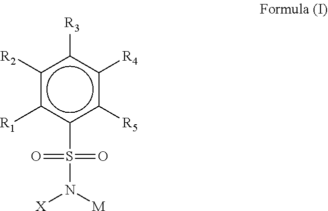

wherein R₁, R₂, R₃, R₄, and R₅, X, and M are defined herein. The compositions described may be used to treat and/or prevent a variety of infections, including bacterial infections, viral infections, fungal infections, and the like. The compositions can be internally administered to a patient via at least one of: oral administration, pulmonary administration, subcutaneous administration, sublingual administration, ocular administration, otic administration, intravenous administration, inter-dermal administration, epidural administration, intraperitoneal administration, and intramuscular administration.

### CROSS-REFERENCE TO RELATED APPLICATIONS

This application claims priority to U.S. Provisional Patent Application Ser. No. 63/227,014, filed on Jul. 29, 2021, which is incorporated by reference in its entirety.

### BACKGROUND

The present disclosure relates to treating and/or preventing medical conditions, such as an infection, which affect the internal pathophysiology of a patient by using a composition comprising a halo active aromatic sulfonamide compound. The methods and compositions disclosed herein find particular application in the treatment of patients by internal administration.

Infections occur when an organism gets access to inter-cellular and intra-cellular regions underneath the outer-protective membranes of the skin. Punctures, injections, bites, cuts, wounds, surgery, splits between skin and mucous membranes, organ or tissue transplants, among others, are all examples which could lead to infection. Infections, such as bacterial infections, viral infections, and fungal infections, can lead to serious and life-threatening conditions. If not addressed, infections can grow, spread, and lead to secondary complications.

Currently, treatments for infections such as viral, bacterial, and fungal infections, include supportive drug therapies like vaccines, antivirals, antibiotics, antibacterials, and antifungals. Depending on the type, source, and degree of infection, treatments and outcomes can vary drastically. Further, certain pathogens are becoming drug-resistant, and thus traditional pharmaceuticals may no longer be effective in treating such infections. The threat of, for example, antibiotic-resistant bacteria to the world's population is serious and growing. Thus, it would be desirable to provide compositions that could treat a wide range of infections, including those which have become resistant to one or more traditional pharmaceuticals, so as to provide greater certainty of immediate effective treatment.

### BRIEF DESCRIPTION

Disclosed herein are compositions useful for the treatment and/or prevention of infections that affect the internal pathophysiology of a patient. In some particular embodiments, these compositions are administered via internal administration to a patient, and in other particular embodiments these compositions are administered via external administration. The compositions comprise an effective amount of a halo active aromatic sulfonamide compound having the structure of Formula (I):

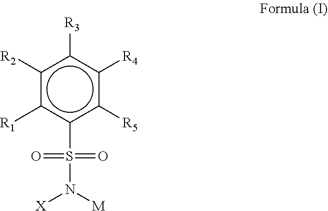

wherein R₁, R₂, R₃, R₄, and R₅, X, and M are defined herein.

The compositions described may be used to treat and/or prevent a variety of infections, including bacterial infections, viral infections, fungal infections, and the like. The compositions can be internally administered to a patient via at least one of: oral administration, pulmonary administration, subcutaneous administration, sublingual administration, ocular administration, otic administration, intravenous administration, inter-dermal administration, epidural administration, intraperitoneal administration, and intramuscular administration. In some embodiments, the compositions can be taken intrathecally, nasally, opthalmically, rectally, topically, enterally, transdermally, buccally, vaginally, or intraurethrally. Further, the compositions may be internally administered as part of a medical device or instrument.

In accordance with one aspect of the present disclosure, a composition for administration to a patient is provided that is pharmaceutically effective for the treatment and/or prevention of an internal infection in the patient, wherein the composition comprises at least a halo active aromatic sulfonamide compound. The halo active aromatic sulfonamide compound may have a structure according to Formula (I) as described above, or may have a structure according to Formula (II):

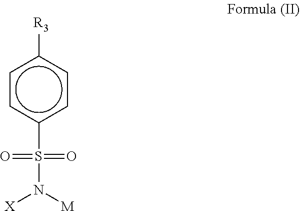

wherein R₃ is COOR′;
R′ is hydrogen, an alkali metal, an alkaline earth metal, substituted C₁-C₁₂ alkyl, unsubstituted C₁-C₁₂ alkyl, substituted aromatic, or unsubstituted aromatic;
X is halogen; and
M is an alkali or alkaline earth metal.

In certain embodiments, the compositions may comprise a halo active aromatic sulfonamide compound having a structure according to Formula (III):

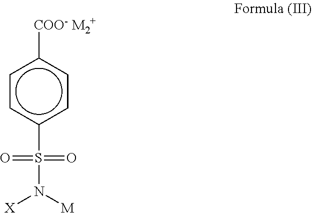

wherein M₂ is hydrogen, an alkali metal, or an alkali earth metal;
X is halogen; and
M is independently an alkali or alkaline earth metal.

In accordance with a further aspect of the present disclosure, a drug delivery device is provided for administering a composition comprising a halo active aromatic sulfonamide compound. The delivery device may be suitable and/or adapted for internal administration of the composition. In specific embodiments, the delivery device may be an inhaler containing an amount of the composition and/or a nebulizer containing an amount of the composition. In other embodiments, the delivery device may be: a time-release device that delivers the composition at a pre-determined time(s) and/or over a pre-determined time(s); a fusion-rate release device that delivers the composition at a pre-determined rate(s); a delayed released device that delivers the composition after a certain pre-determined about of time(s), and/or any combination thereof.

In accordance with an additional aspect of the present disclosure, provided is a method for treating a patient suffering from an infection or is at risk of developing an infection using a composition. The method can include at least the steps of: determining a dosage of the composition; and internally administering the dosage of the composition to the patient, wherein the composition comprises a halo active aromatic sulfonamide compound of Formula (I):

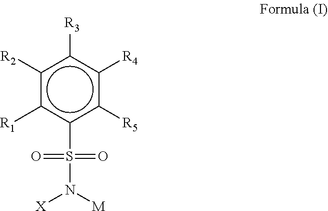

wherein R₁, R₂, R₃, R₄, and R₅ are independently selected from hydrogen, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic;
R′ is hydrogen, an alkali metal, an alkaline earth metal, substituted C₁-C₁₂ alkyl, or unsubstituted C₁-C₁₂ alkyl; and
R″ is hydrogen or substituted or unsubstituted C₁-C₁₂ alkyl, where the two R″ groups in CON(R″)₂ and N(R″)₂ may be independently selected;
X is halogen; and
M is an alkali or alkaline earth metal.

These and other non-limiting features or characteristics of the present disclosure will be further described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a graph showing the averaged amount of MRSA found in a wound of control (untreated) specimen and experimental (treated) specimen at 24 hours after infection.

FIG. 2 is a graph showing the averaged amount of MRSA found in a wound, the spleen, and the liver of control (untreated) specimen and experimental (treated) specimen at 48 hours after infection.

FIG. 3 is a flowchart illustrating a method of treating a patient in accordance with one aspect of the present disclosure.

### DETAILED DESCRIPTION

A more complete understanding of the components, processes, and apparatuses disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Halo active aromatic sulfonamide organic compounds have been known in general to reduce or eliminate odor. Chloramine-T is an example of a sulfonamide organic compound which has been used in many applications. The usefulness of chloramine-T is predicated on its ability to release an active chloride ion when needed on demand, immediately after which it simultaneously generates an active aromatic sulfo nitrene companion ion. The chlorine atom has a +1 formal charge in a hypochlorite ion, ClO⁻, which is the form taken by the chlorine atom when dissociated from the sulfonamide compound. Reference to the chlorine atom as having a +1 or 1⁻ charge may be used in this application interchangeably because this terminology has no effect on the compound itself or its use.

It has been found that halo active aromatic sulfonamide organic compounds can also have an antimicrobial effect that can extend over long periods of time when applied to surfaces in places such as a hospital, nursing home or long-term care facility, school, jail or prison, a vehicle, a house, a gym or workout facility, or a supermarket. Compared with typical disinfectants such as bleach, hydrogen peroxide, and peracetic acid, hydrates of halo active aromatic sulfonamide organic compounds continue to exhibit disinfectant ability over long time periods, such as over 24 hours, over 48 hours, over 72 hours, or even as long as 168 hours. Furthermore, such disinfectants are generally caustic to bodily tissues.

It has now been found that halo active aromatic sulfonamide organic compounds and compositions containing such compounds can obtain surprisingly effective antimicrobial results when administered to a patient in order to treat or prevent infections that affect the external pathophysiology of the patient. Furthermore, it has been found that such compositions and administrations provide acceptable caustic effect to body tissues of humans, mammals, and other animals, thereby allowing for effective pharmaceutical application.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

### Definitions

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 to 10” is inclusive of the endpoints, 2 and 10, and all the intermediate values).

The term “about” can be used to include any numerical value that can vary without changing the basic function of that value. When used with a range, “about” also discloses the range defined by the absolute values of the two endpoints, e.g. “about 2 to about 4” also discloses the range “from 2 to 4.” The term “about” may refer to plus or minus 10% of the indicated number.

The term “ambient temperature” refers to a temperature of 20° C. to 25° C.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, the aldehyde group —CHO is attached through the carbon of the carbonyl group.

The term “alkyl” refers to a radical composed entirely of carbon atoms and hydrogen atoms which is fully saturated. The alkyl radical may be linear, branched, or cyclic, and such radicals may be referred to as linear alkyl, branched alkyl, or cycloalkyl.

The term “aromatic” refers to a radical that has a ring system containing a delocalized conjugated pi system with a number of pi-electrons that obeys Hückel's Rule. The ring system may include heteroatoms (e.g. N, S, Se, Si, O), or may be composed exclusively of carbon and hydrogen. Exemplary aromatic groups include phenyl, thienyl, naphthyl, and biphenyl. The term “aromatic”, as used herein, does not refer to a smell detected by the nose.

The term “aryl” refers to an aromatic radical composed exclusively of carbon and hydrogen. Exemplary aryl groups include phenyl, naphthyl, and biphenyl.

The term “heteroaryl” refers to an aromatic radical containing at least one heteroatom. Exemplary heteroaryl groups include thienyl. Note that “heteroaryl” is a subset of “aromatic”, and is exclusive of “aryl”.

The term “alkoxy” refers to an alkyl radical which is attached to an oxygen atom, i.e. —O—CₙH₂ₙ₊₁, to a molecule containing such a radical.

The term “halogen” refers to fluorine, chlorine, bromine, and iodine.

The term “substituted” refers to at least one hydrogen atom on the named radical being substituted with another functional group, such as halogen, —CN, or —NO₂. Besides the aforementioned functional groups, an aromatic group may also be substituted with alkyl or alkoxy. An exemplary substituted aryl group is methylphenyl.

The term “alkali metal” refers to lithium, sodium, and potassium.

The term “alkaline earth metal” refers to magnesium and calcium.

The term “body” means the body of humans and/or animals; the term “subject” and “patient” means the body of such a human and/or animal.

As used herein, when referring to a “microbial,” “microbe,” or “pathogen,” the term may refer to any of a virus, a bacteria, a spore, a fungi, or the like, which may cause an infection in a human and/or animal.

Unless otherwise indicated, the term “infection” is intended to encompass any type of infection, such as a bacterial infection, viral infection, fungal infection, and the like.

As used herein, when referring to “internal administration” of a composition, the term refers to the administration of the composition into the subject, as opposed to onto an external surface of the subject.

The terms “treatment” and “treating” are intended to encompass also prophylaxis, therapy, and cure. Accordingly, in one aspect, a treatment involves preventing or delaying or slowing the onset of a condition, disease, or disorder (e.g. the symptoms associated with the disease, condition, or disorder). In another aspect, a treatment involves treating (e.g. minimizing or reducing or slowing the development or reversing) an existing condition, disease, or disorder (e.g. the symptoms associated with the disease, condition, or disorder). In one embodiment, a treatment provides a cure for a condition, disease, or disorder (e.g. eliminates the condition, disease, or disorder).

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject copolymer and/or composition from one organ, or portion of the body, to another organ, or portion of the body. Each carrier must be “acceptable” in the sense of being compatible with the other ingredients of the formulation and not unduly injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutically effective amount”, as defined herein, refers to an amount of the sulfonamide that is capable of providing therapeutic effect to the patient when used. This amount may be measured in absolute amounts (e.g. grams) or in relative amounts (e.g. mg/kg).

Compositions

Disclosed in various embodiments herein are compositions for administration to a patient and effective for the treatment or prevention of an internal condition, such as an infection in the patient. The compositions of the present disclosure comprise (A) a halo active aromatic sulfonamide compound, as described further herein. The compositions can also include additional components, including a buffering agent (B), a surfactant (C), and a solvent (D), as well as others, and any combination of these additional ingredients.

Halo Active Aromatic Sulfonamide Compound

The halo active aromatic sulfonamide compounds (A) disclosed herein are stable and do not decompose in aqueous solution, allowing the composition to have a long shelf life and persistent therapeutic effect. The halo active aromatic sulfonamide compounds are also very soluble in water, low in toxicity, and have minimal bleach odor.

In general, the halo active aromatic sulfonamide compound is present in the composition in the amount of about 0.0001 wt % to about 100 wt %, including from about 0.0001 wt % to about 0.001 wt %, from about 0.001 wt % to about 0.01 wt %, from about 0.01 wt % to about 0.1 wt %, from about 0.1 wt % to about 1 wt %, from about 1 wt % to about 5 wt %, from about 5 wt % to about 10 wt %, from about 10 wt % to about 15 wt %, from about 15 wt % to about 20 wt %, from about 20 wt % to about 25 wt %, from about 25 wt % to about 30 wt %, from about 30 wt % to about 35 wt %, from about 35 wt % to about 40 wt %, from about 40 wt % to about 45 wt %, from about 45 wt % to about 50 wt %, from about 50 wt % to about 55 wt %, from about 55 wt % to about 60 wt %, from about 60 wt % to about 65 wt %, from about 65 wt % to about 70 wt %, from about 70 wt % to about 75 wt %, from about 80 wt %, from about 80 wt % to about 85 wt %, from about 85 wt % to about 90 wt %, from about 90 wt % to about 95 wt %, and from about 95 wt % to about 100 wt %, including any combination of endpoints thereof. In some embodiments, the halo active aromatic sulfonamide compound is present in the composition in the amount of about 0.0001 wt % to about 45 wt %, including from about 0.0001 wt % to about 30 wt %, from about 0.0001 wt % to about 25 wt %, from about 0.0001 wt % to about 20 wt %, from about 0.1 wt % to about 20 wt %, from about 0.0001 wt % to about 5 wt %, from about 1 wt % to about 2 wt %, from about 2 wt % to about 3 wt %, from about 3 wt % to about 4 wt %, and from about 4 wt % to about 5 wt %. In particular embodiments, the composition may consist essentially of the halo active aromatic sulfonamide compound.

In some embodiments, the composition comprises from about 0.021 wt % to about 0.085 wt % of the halo active aromatic sulfonamide compound, or from about 0.085 wt % to about 0.17 wt % of the halo active aromatic sulfonamide compound. In other embodiments, the composition may comprise from about 210 milligrams (mg) to about 850 milligrams of the halo active aromatic sulfonamide compound per liter of composition, or from about 850 milligrams to about 1700 milligrams of the halo active aromatic sulfonamide compound per liter of the composition. In some embodiments, the dosage of the composition is such that the total amount of the halo active aromatic sulfonamide compound administered to the patient is at least 100 milligrams per kilogram (kg) of patient weight, or at most 460 milligrams per kilogram of patient weight. In still further embodiments, the composition may comprise at least 0.021 wt % of the halo active aromatic sulfonamide compound, or at least 0.085 wt % of the halo active aromatic sulfonamide compound, or at least 0.17 wt % of the halo active aromatic sulfonamide compound, or at least 210 milligrams of the halo active aromatic sulfonamide compound per liter of the composition, or at least 850 milligrams of the halo active aromatic sulfonamide compound per liter of the composition, or at least 1700 milligrams of the halo active aromatic sulfonamide compound per liter of the composition.

The halo active aromatic sulfonamide compound used in the compositions of the present disclosure can have the structure of base Formula (I):

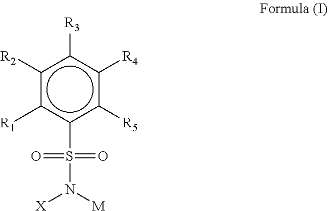

wherein R₁, R₂, R₃, R₄, and R₅ are independently selected from hydrogen, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic;
R′ is hydrogen, an alkali metal, an alkaline earth metal, substituted C₁-C₁₂ alkyl, or unsubstituted C₁-C₁₂ alkyl; and
R″ is hydrogen or substituted or unsubstituted C₁-C₁₂ alkyl, where the two R″ groups in CON(R″)₂ and N(R″)₂ may be independently selected;
X is halogen; and
M is an alkali or alkaline earth metal.

Generally, M is sodium or potassium. X is generally chlorine, bromine, fluorine, or iodine, and in particular embodiments is chlorine. Compounds of Formula (I) may or may not be hydrated. In particular embodiments, the compounds of Formula (I) are a trihydrate (i.e., n=3) or a hexahydrate (i.e. n=6). In other embodiments, the compound is in a solid form, such as a powder.

When the phenyl and/or alkyl group is substituted, one or more hydrogen atoms may be independently replaced with hydroxyl or halogen.

In particular embodiments of Formula (I), R₃ is methyl, COOH, or COOK; R₁, R₂, R₄, and R₅ are independently selected from hydrogen, COOH, COOK, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic; X is halogen; and M₁ is an alkali or alkaline earth metal.

In further embodiments, R₃ is methyl, COOH, or COOK; R₁, R₂, R₄, and R₅ are independently selected from hydrogen, COOH, COOK, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic; X is halogen; M is an alkali or alkaline earth metal; and at least one of R₁, R₂, R₄, and R₅ is not hydrogen.

In yet other embodiments of Formula (I), R₃ is selected from COOH, COOK, COOR′, CON(R″)₂, CN, NO₂, halogen, and substituted or unsubstituted C₂-C₁₂ alkyl; R₁, R₂, R₄, and R₅ are independently selected from hydrogen, COOH, COOK, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic; X is halogen; and M is an alkali or alkaline earth metal.

In still other embodiments of Formula (I), R₁, R₂, R₃, R₄, and R₅ are independently selected from hydrogen, COOH, COOK, NO₂, halogen, N(R″)₂, substituted or unsubstituted C₁-C₁₂ alkyl, and substituted or unsubstituted aromatic; X is halogen; and M is an alkali or alkaline earth metal.

In yet other embodiments of Formula (I), R₂ and R₄ are identical to each other; and R₁, R₃, and R₅ are hydrogen.

In yet other embodiments of Formula (I), R₂ and R₄ are hydrogen; and R₁, R₃, and R₅ are identical to each other.

In more specific embodiments of Formula (I), R₃ is selected from COOH, COOK, COOR′, and CON(R″)₂. Most desirably, R₃ is COOH or COOK, while R₁, R₂, R₄, and R₅ are hydrogen.

In other embodiments of Formula (I), R₁, R₂, R₃, R₄, and R₅ are independently selected from hydrogen, COOH, COOK, COOR′, CON(R″)₂, NO₂, halogen, N(R″)₂, substituted or unsubstituted C₁-C₁₂ alkyl, and substituted or unsubstituted aromatic; wherein at least one of R₁, R₂, R₃, R₄, and R₅ is not hydrogen; X is halogen; and M is an alkali or alkaline earth metal.

In still other embodiments of Formula (I), R₃ is COOH or COOM₁; R₁, R₂, R₄, and R₅ are independently selected from hydrogen, NO₂, halogen, N(R″)₂, substituted or unsubstituted C₁-C₁₂ alkyl, and substituted or unsubstituted aromatic; X is halogen; and M is an alkali or alkaline earth metal. In further specific embodiments, at least one of R₁, R₂, R₄, and R₅ is not hydrogen.

In some embodiments of Formula (I), at least one of R₁, R₂, R₃, R₄, or R₅ are not hydrogen. In more specific embodiments of Formula (I), at least two of R₁, R₂, R₃, R₄, or R₅ are not hydrogen. In other words, the benzene ring contains the sulfonamide substituent and an additional one or two other substituents.

In other embodiments of Formula (I), the halo active aromatic sulfonamide compound has the structure of Formula (II):

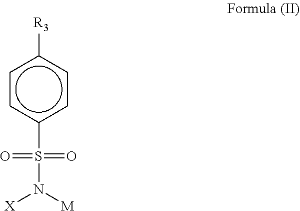

wherein R₃ is COOR′; R′ is hydrogen, an alkali metal, an alkaline earth metal, substituted C₁-C₁₂ alkyl, unsubstituted C₁-C₁₂ alkyl, substituted aromatic, or unsubstituted aromatic; X is halogen; and M is an alkali or alkaline earth metal. The N-chloro-4-carboxybenzenesulfonamide compound of Formula (II) is also referred to herein as BENZ. BENZ exhibits a lower chlorine smell than chloramine-T or chloramine-B. BENZ is also known as Monalazone Disodium, CAS #61477-95-0.

One particular sulfonamide compound contemplated for use is N-chloro-4-carboxybenzenesulfonamide (i.e. BENZ). This compound is shown below as Formula (III):

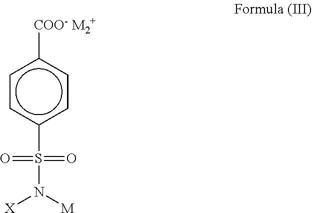

wherein M₂ is hydrogen, an alkali metal, or an alkali earth metal; X is halogen; and M is independently an alkali or alkaline earth metal. Desirably, M₂ is hydrogen, sodium, or potassium.

In other embodiments, one or more of R₁, R₂, R₃, R₄, and R₅ are substituted with —COOR′ (and the others are hydrogen). In this regard, it is believed that when the halo active aromatic sulfonamide compound has two or more ionic charges, that the compound has higher antimicrobial performance.

In further embodiments, the composition may comprise a halo active aromatic sulfonamide compound that is a disodium salt. That is, the halo active aromatic sulfonamide compound may have the general structure of Formula (I) wherein the functional groups R₁, R₂, R₃, R₄, and R₅ contain two sodium atoms that dissociate upon hydration.

In still further embodiments, the composition may comprise a halo active aromatic sulfonamide compound that is a trisodium salt. That is, the halo active aromatic sulfonamide compound may have the general structure of Formula (I) wherein the functional groups R₁, R₂, R₃, R₄, and R₅ contain three sodium atoms that dissociate upon hydration.

In some embodiments, the composition may comprise a halo active aromatic sulfonamide compound that is a dicarboxy sulfonamide. That is, the halo active aromatic sulfonamide compound may have the general structure of Formula (I) wherein two of the functional groups R₁, R₂, R₃, R₄, and R₅ are —COOH.

In particular embodiments, the composition may be formulated for improved fat solubility. That is, in certain embodiments, the composition comprises a halo active aromatic sulfonamide compound wherein at least one of R₁, R₂, R₃, R₄, or R₅ is a long chain aliphatic group, such as a substituted or unsubstituted C₁-C₁₂ alkyl.

It is also believed that the compounds of Formula (I) do not have mutagenic properties, which may reduce the evolution of resistance to these compounds. Alternatively, their use might lower the total microbial load which is encountered by antibiotics used in conjunction therewith.

One significant difference between the compounds of Formula (I) and conventional sulfa drugs is the presence of a stable halogen on the nitrogen atom of the sulfonamide group, which provides a higher efficacy per mole than any sulfa drug. This results in much quicker kill without the side effects of typical halogen chemistry. In addition, the sulfonamide compound selectively kills bacteria, viruses, and fungi.

One of ordinary skill in the art would not have expected such sulfonamide compounds to be effective for internal use as a pharmaceutical product because prior uses of such compounds were almost always labeled with a warning not to ingest it.

Additional Components

The compositions of the present disclosure may include or exclude one or more additional components, including, for example, at least one of a buffering agent (B), a surfactant (C), and a solvent (D), among others.

A buffering agent (B) can be included to maintain the composition within a desired pH range. In particular embodiments, the composition may have a pH of from about 6 to about 10. In specific embodiments, the composition may have a pH of about 6, or about 6.5, or about 7, or about 7.5, or about 8, or about 8.5, or about 9, or about 9.5, or about 10. The buffering agent can be added up to the limit of solubility in the compositions. In particular embodiments, the preferred weight ratio of the sulfonamide compound to the buffering agent is from about 50:1 to about 1:1, or from about 50:1 to about 2:1, or from about 20:1 to about 2:1.

Exemplary buffering agents include sodium bicarbonate, potassium bicarbonate, sodium carbonate, potassium carbonate, acetate buffers (such as sodium acetate), phosphate buffers (such as tri and di sodium phosphate and mixtures thereof, pH blended phosphates, sulfate buffers (such as di and tri sodium sulfate), and mixtures thereof. The preferred buffering agent is sodium bicarbonate.

A surfactant (C), or wetting agent, can also be added to the composition. The surfactant decreases surface tension, allowing the sulfonamide compound to be move more freely upon administration. Both non-ionic and anionic surfactants can be used. However, in some specific embodiments, a surfactant is not used. The surfactant can be present in the composition in the amount of about 0.0001 wt % to about 5 wt %.

Additionally, a solvent (D), which acts as a carrier vehicle for internally administering the halo active aromatic sulfonamide compound to a patient, may be included. In particular embodiments, the solvent can make up the majority of the composition. That is, the compositions may comprise a balance amount of the solvent (i.e. comprise the remaining portion of the composition). In specific embodiments, the solvent may be water, a pharmaceutically-acceptable carrier, or a combination thereof.

The composition may also include excipients and adjuvants as desired. Particular excipients include buffering agents, surfactants, preservative agents, bulking agents, polymers, and stabilizers. Buffering agents are used to control the pH of the composition. Surfactants are used to stabilize proteins, inhibit protein aggregation, inhibit protein adsorption to surfaces, and assist in protein refolding. Exemplary surfactants include Tween 80, Tween 20, Brij 35, Triton X-10, Pluronic F127, and sodium dodecyl sulfate. Preservatives are used to prevent microbial growth. Examples of preservatives include benzyl alcohol, m-cresol, and phenol. Bulking agents are used during lyophilization to add bulk. Hydrophilic polymers such as dextran, hydroxyl ethyl starch, polyethylene glycols, and gelatin can be used to stabilize proteins. Polymers with nonpolar moieties such as polyethylene glycol can also be used as surfactants. Protein stabilizers can include polyols, sugars, amino acids, amines, and salts. Suitable sugars include sucrose and trehalose. Amino acids include histidine, arginine, glycine, methionine, proline, lysine, glutamic acid, and mixtures thereof. Proteins like human serum albumin can also competitively adsorb to surfaces and reduce aggregation. It should be noted that particular compounds can serve multiple purposes. For example, histidine can act as a buffering agent and an antioxidant. Glycine can be used as a buffering agent and as a bulking agent. Adjuvants may be used to improve immune response. Examples of adjuvants may include aluminum salts, AS04, MF59, AS01B, and CpG 1018.

The dosage form of the composition may vary. For example, the dosage form may be an aerosol, a capsule, an emulsion, a film, a gel, granules, a gum, an injection, a lozenge, a paste, pellets, a pill, a powder, a solution, a spray, a suppository, a suspension, or a tablet. These dosage forms are especially suitable for internal administration, such as within a body cavity of the patient or user like the mouth, nose, or rectum.

In an aerosol, the composition is packaged under pressure and also contains a propellant. When actuated, a dose of the composition is delivered in the form of a plume or mist of particles/droplets, or in other words in dry or wet form. For example, the composition may be sprayed upon the skin. The composition may be a liquid in the form of a suspension or solution, or can be dry solids such as particles. Examples of propellants can include nitrous oxide, nitrogen (N2), carbon dioxide, HFA 134a, HFA 227, certain hydrocarbons like propane or butane, ethers, and combinations thereof. In contrast, a spray is not pressurized, and the plume or mist is generated by discharge through the nozzle. The composition is usually a liquid in the form of a suspension or solution. The aerosol or spray may be inhaled through the mouth or the nose, or can be sprayed sublingually.

A capsule or pellet is a solid dosage form in which the composition is enclosed within a soluble container or shell. The shell may be a single piece, or may be made from two pieces. The shell can be hard or soft. The capsule can be made for immediate release of the composition, or can be a modified-release capsule. For example, the capsule may be coated with an enteric coating to protect the capsule from the gastric environment or may be modified as an extended-release capsule that releases the composition over an extended time period. The shell is made, for example from a gelatin or a cellulose polymer. Capsules and pellets are typically ingested.

An emulsion includes two immiscible liquid phases that are usually stabilized with one or more suitable emulsifying agents. Those two phases may include an aqueous phase and an organic phase, with the sulfonamide (A) being present in one of the phases. The organic phase of an emulsion can be formed from oils (mineral and/or vegetable), fatty alcohols, fatty acids, and/or fatty esters. Emulsifying agents can include surfactants, which can be non-ionic, cationic, anionic, or zwitterionic. Emulsions can be taken orally or parenterally, and can be injectable.

A film is a thin sheet that can be taken orally, buccally, or sublingually. The film may contain one or more layers, and can be formulated with edible polymers or with water-soluble polymers. The dissolution rate of the film can be controlled.

A gel can be solid or semisolid, but is mostly liquid. A gel contains a gelling agent, such as starch, xanthan, guar gum, locust bean gum, gum karaya, gum tragacanth, gum Arabic, alginate, pectin, carrageenan, gelatin, gellan agar, methylcellulose, hydroxymethylcellulose, and/or carboxymethylcellulose. Other ingredients can include sugars, water, sweeteners, and flavoring agents. A paste is similar to a gel, and usually contains a high percentage of small, dispersed solids as well. Gels/pastes can be molded into a desired shape for oral delivery, and are colloquially called “gummies”.

Granules are a solid formed by agglomeration of smaller particles, and can also include excipients, binders, and/or solvents. They are typically taken orally.

A gum is a dosage form that is intended to be chewed rather than swallowed. They typically include a gum base, as well as plasticizers, softeners, and/or sugars.

An injection is typically a liquid form, such as a solution or suspension, which is intended to be injected into the body. A solution includes the sulfonamide (A) in a liquid form. A solution can be ingested, injected, or inhaled. A suspension consists of solid particles dispersed in a liquid phase. A suspension can ingested, injected, inhaled, or taken via ophthalmic and otic routes.

A lozenge is a solid form that is designed to dissolve or disintegrate slowly, and is typically taken orally. A lozenge typically is molded and includes gelatin, sugars, or other bases. A pill is a solid and is typically distinguished from capsules only by its manufacturing process. A pill is usually prepared from a wet mass which is molded. A pill is usually taken orally. A powder is a solid (i.e. the sulfonamide (A)) in a finely divided state. A suspension typically consists of solid particles (i.e. the sulfonamide (A)) dispersed in a liquid phase.

A suppository is intended to be taken rectally. The suppository typically includes a base such as cocoa butter gelatin, vegetable oils, or mixtures of various polymers such as polyethylene glycol.

A tablet is a solid typically made by compaction of powders or granules. Tablets can also be coated with an enteric coating or an extended-release coating. Tablets can be taken buccally, orally, chewed, or sublingually.

While not being limited by theory, it is believed the antimicrobial kill performance of the sulfonamide will extend over that time period as well, so that new growths of microorganisms will also be eliminated. Extended treatment and prophylactic protection of patients is thus possible. Further, unlike other products such as bleach, which should not be used within a human or animal body, the present compositions may be internally administered to a patient because they surprisingly have minimal effects on healthy tissues.

In accordance with certain aspects of the present disclosure, a delivery device is provided that can comprise the composition described above. The delivery device may be suitable and/or adapted for internal administration of the composition. In specific embodiments, the delivery device may be an inhaler containing an amount of the composition and/or a nebulizer containing an amount of the composition. In further embodiments, the delivery device may be: a time-release device that delivers the composition at a pre-determined time(s) and/or over a pre-determined time(s); a fusion-rate release device that delivers the composition at a pre-determined rate(s); a delayed released device that delivers the composition after a certain pre-determined about of time(s), and/or any combination thereof.

In still further embodiments, the delivery device may be one or more of a medical device, a medical instrument, an implant, and the like. That is, the compositions disclosed herein may be incorporated into at least one of a medical device, a medical instrument, and/or an implant that is then internally administered to the patient. For example, the composition may be incorporated into a medical instrument including, but not limited to, at least one of a syringe, a catheter, an IV set, surgical gloves, gauze, wound dressing, a dialysis machine, a medical scope, and the like, which is then contacted with a portion of the inside of the patient's body (e.g. transcutaneously). The composition may also be incorporated into a medical device, implant, or prosthetic, including but not limited to, organic implants (e.g. skin, bone, or other body tissues), metal implants, plastic and polymer implants, ceramic implants, and the like. The composition may be incorporated into such medical instruments, devices, implants, and/or prosthetics at least by sterilization prior to, during, and/or after use of the instrument, device, implant, or prosthetic. That is, the medical device, medical instrument, medical implant, or medical prosthetic, or a portion thereof, may include or be coated with the compositions disclosed herein.

Some conditions which affect the internal pathophysiology can be treated by external administration. For example, many medications can be ingested or applied to the skin to obtain the same therapeutic effect, such as hormonal therapies, pain medications, etc. It is thus contemplated that the compositions of the present disclosure may be provided for external administration as well. Examples of some dosage forms which may be applied externally to affect the internal pathophysiology include an aerosol, a cream, an emulsion, a gel, a lotion, an ointment, a paste, a powder, a soap, a spray, a suspension, or a tape. These dosage forms are especially suitable for external administration, such as upon the skin of the patient or user.

An aerosol/spray, emulsion, gel, paste, powder, and suspension have been previously described. These dosage forms can also be applied topically to the skin.

Creams, lotions, and ointments are different forms of emulsions. A cream is semisolid, and typically contains more than 20 wt % water and less than 50 wt % of hydrocarbons, waxes, or polyols. A lotion is very similar to a cream, and is generally distinguished by being more fluid (i.e. a lower viscosity). An ointment is semisolid, and typically contains less than 20 wt % water and more than 50 wt % of hydrocarbons, waxes, or polyols. The organic phase of an emulsion can be formed from oils (mineral and/or vegetable), fatty alcohols, fatty acids, and/or fatty esters. Emulsifying agents can include surfactants, which can be non-ionic, cationic, anionic, or zwitterionic. A soap is essentially a solid emulsion.

A tape includes a substrate into which the sulfonamide (A) is impregnated. The substrate can be, for example, a woven or non-woven material. One side of the substrate is coated with an adhesive agent, which holds the tape in place without the need for additional material. A tape is also known as a bandage or a patch.

Methods

In accordance with still another aspect of the present disclosure, provided are methods for treating a patient with a composition, wherein the patient is suffering from an infection or is at risk of developing an infection. For example, the patient may have recently undergone surgery or had a transplant, may be about to undergo surgery, have a transplant, or have a medical device implanted, or may have already contracted a type of infection, including but not limited to, a viral infection, a bacterial infection, a fungal infection, and/or the like. In some embodiments, the patient may be infected by or at risk of developing an infection from at least one of: human immunodeficiency virus (HIV), hepatitis; Pseudomonas; methicillin-resistant Staphylococcus aureus (MSRA); vancomycin-resistant enterococci (VRE); Streptococcus; Staphylococcus; Escherichia coli (E. coli); Klebsiella; Salmonella; Clostridium difficile (C. diff.), Candida; Acinetobacter baumannii; and influenza. In particular embodiments, the infection source (i.e. virus, bacterial, fungi, etc., that is the cause of infection) may be a drug-resistant infection. The disease that is being treated could be a blood disorder, a parasite, or a type of cancer. Further, the patient may be, for example, a mammal including but not limited to a human or an animal.

In some particular embodiments, the compositions can be internally administered to a patient. As used herein, the terms “internal” and “internally” refer to the inside of the patient's body. That is, treatment using the disclosed compositions may be achieved via oral administration, pulmonary administration, subcutaneous administration, sublingual administration, ocular administration, otic administration, intravenous administration, inter-dermal administration, epidural administration, intraperitoneal administration, intramuscular administration, intrathecally, nasally, opthalmically, rectally, topically, enterally, transdermally, buccally, vaginally, or intraurethrally, and the like. Further, treatment using the disclosed compositions may be achieved via contact with a medical device, or the like, which contains the composition. For example, the medical device may be coated with the composition and then implanted or otherwise contacted with an internal portion of the patient's body. In other embodiments, the composition is externally administered, which treats the internal condition.

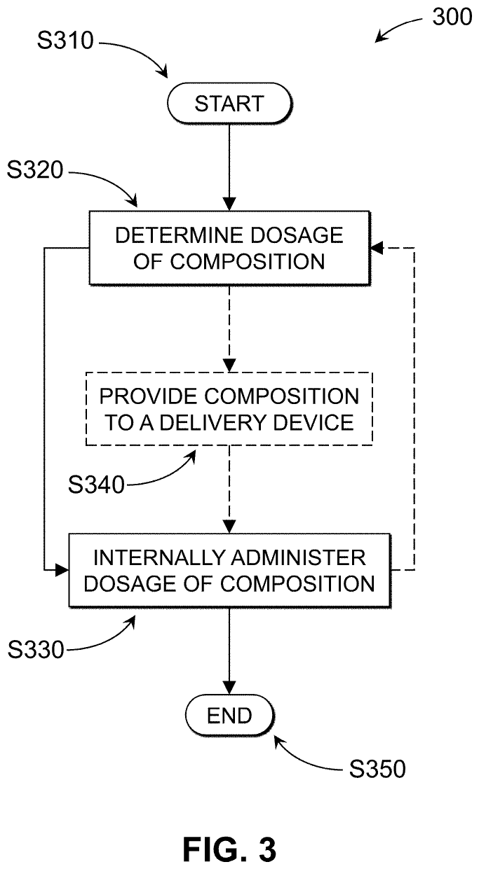

With reference to FIG. 3, the methods 300 disclosed herein can, beginning at a step S310, include the steps of: (1) determining a dosage of a composition S320; and (2) internally administering the dosage of the composition to the patient S330.

In particular embodiments, the composition comprises a halo active aromatic sulfonamide compound having the structure of Formula (I):

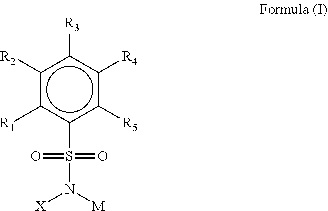

wherein R₁, R₂, R₃, R₄, and R₅ are independently selected from hydrogen, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic;
R′ is hydrogen, an alkali metal, an alkaline earth metal, substituted C₁-C₁₂ alkyl, or unsubstituted C₁-C₁₂ alkyl; and
R″ is hydrogen or substituted or unsubstituted C₁-C₁₂ alkyl, where the two R″ groups in CON(R″)₂ and N(R″)₂ may be independently selected;
X is halogen; and
M is an alkali or alkaline earth metal.

In a step S320, a pharmaceutically effective amount of the composition for treating the underlying condition of the patient may be determined. The pharmaceutically effective amount of the composition, or the dosage, may be dependent on a variety of factors, including but not limited to, the patient's weight. Thus, in some embodiments, the step S320 of determining a dosage of the composition may include determining at least a weight of the patient. In further embodiments, the total amount of the composition administered to the patient may include from about 0.5 milligrams to about 100 grams of the halo active aromatic sulfonamide compound, such as from about 0.1 mg to about 0.2 mg, from about 0.2 mg to about 0.3 mg, from about 0.3 mg to about 0.4 mg, from about 0.4 mg to about 0.5 mg, from about 0.5 mg to about 0.6 mg, from about 0.6 mg to about 0.7 mg, from about 0.7 mg to about 0.8 mg, from about 0.8 mg to about 0.9 mg, from about 0.9 mg to about 1 mg, from about 1 mg to about 2 mg, from about 2 mg to about 3 mg, from about 3 mg to about 4 mg, from about 4 mg to about 5 mg, from about 5 mg to about 6 mg, from about 6 mg to about 7 mg, from about 7 mg to about 8 mg, from about 8 mg to about 9 mg, from about 9 mg to about 10 mg, from about 10 mg to about 20 mg, from about 20 mg to about 30 mg, from about 30 mg to about 40 mg, from about 40 mg to about 50 mg, from about 50 mg to about 60 mg, from about 60 mg to about 70 mg, from about 70 mg to about 80 mg, from about 80 mg to about 90 mg, from about 90 mg to about 100 mg, from about 100 mg to about 200 mg, from about 200 mg to about 300 mg, from about 300 mg to about 400 mg, from about 400 mg to about 500 mg, from about 500 mg to about 600 mg, from about 600 mg to about 700 mg, from about 700 mg to about 800 mg, from about 800 mg to about 900 mg, from about 900 mg to about 1 grams, from about 1 grams to about 5 grams, from about 5 grams to about 10 grams, from about 10 grams to about 20 grams, from about 20 grams to about 30 grams, from about 30 grams to about 40 grams, from about 40 grams to about 50 grams, from about 50 grams to about 60 grams, from about 60 grams to about 70 grams, from about 70 grams to about 80 grams, from about 80 grams to about 90 grams, from about 90 grams to about 100 grams, including any combination of endpoints thereof.

In certain embodiments, the compositions may include at least one of a buffering agent, a surfactant, and/or a solvent as described above. In alternative embodiments, the composition may exclude one or more of a buffering agent, a surfactant, and/or a solvent.

As discussed above, the compositions can be internally administered S330 to the patient via oral administration, pulmonary administration, subcutaneous administration, sublingual administration, ocular administration, otic administration, intravenous administration, inter-dermal administration, epidural administration, intraperitoneal administration, intramuscular administration, intrathecally, nasally, opthalmically, rectally, topically, enterally, transdermally, buccally, vaginally, or intraurethrally, and the like.

Further, the composition may be incorporated into a drug delivery device suitable and/or adapted for internal administration S330 of the composition. In specific embodiments, the delivery device may be an inhaler containing an amount of the composition and/or a nebulizer containing an amount of the composition. In further embodiments, the delivery device may be: a time-release device that delivers the composition at a pre-determined time(s) and/or over a pre-determined time(s); a fusion-rate release device that delivers the composition at a pre-determined rate(s); a delayed released device that delivers the composition after a certain pre-determined about of time(s), and/or any combination thereof.

Additionally, the composition may be internally administered S330 to the patient in such a manner to target a specific organ, including but not limited to, at least one of the liver, spleen, colon, intestine, a muscle, kidney, heart, pancreas, gallbladder, lung, and/or the brain.

In some embodiments, the method 300 may include a step of providing a pharmaceutically effective amount of the composition to a delivery device S340. For example, the delivery device may be a medical device, a medical instrument, a medical implant, and/or a prosthetic device that is provided with the composition. In certain embodiments, the delivery device or a portion thereof may be coated with a pharmaceutically effective amount of the composition. Then the delivery device containing a pharmaceutically effective amount of the composition may be internally administered S330 to the patient (e.g. via implantation or otherwise contacted with an internal portion of the patient's body). In particular embodiments, the amount of the composition provided to the delivery device is sufficient to deliver the determined dosage of the composition (i.e. in step S320) to the patient.

The dosage of the composition may also be administered in one or more doses over a period of time. That is, the method may include the additional step of repeating the same or a different dosage of the composition after a period of time. For example, the method of treating a patient suffering from an infection and/or at risk of developing an infection can include administering a first dosage of the composition to the patient, waiting a first period of time, administering a second dosage of the composition to the patient, waiting a second period of time, and so forth, wherein the first and second doses of the composition may be the same or different. In other words, the dosage of the composition may be administered to the patient in two or more doses over a certain period of time (i.e. a treatment period). In particular embodiments, an effective dosage of the disclosed compositions may be administered several times per hour, or about every hour, or about every two hours, or about every four hours, or about every eight hours, or at least once per day, or at least twice per day, or at least once per week, or about one per month. This administration regiment may be repeated a plurality of times over the duration of several hours to several months, or until the treatment and/or prevent of the target indication is completed. The composition can be externally administered or internally administered, as appropriate.

In particular embodiments, for example, depending on the route of administration and the administration regiment, the composition may be delivered such that the halo active aromatic sulfonamide compound is administered at a rate of from about 0.001 mg/kg/hour to about 1000 mg/kg/hour, such as from about 0.001 mg/kg/hour to about 0.01 mg/kg/hour, from about 0.01 mg/kg/hour to about 0.1 mg/kg/hour, from about 0.1 mg/kg/hour to about 1 mg/kg/hour, from about 1 mg/kg/hour to about 10 mg/kg/hour, from about 10 mg/kg/hour to about 20 mg/kg/hour, from about 20 mg/kg/hour to about 30 mg/kg/hour, from about 30 mg/kg/hour to about 40 mg/kg/hour, from about 40 mg/kg/hour to about 50 mg/kg/hour, from about 50 mg/kg/hour to about 60 mg/kg/hour, from about 60 mg/kg/hour to about 70 mg/kg/hour, from about 70 mg/kg/hour to about 80 mg/kg/hour, from about 80 mg/kg/hour to about 90 mg/kg/hour, from about 90 mg/kg/hour to about 100 mg/kg/hour, from about 100 mg/kg/hour to about 150 mg/kg/hour, from about 150 mg/kg/hour to about 200 mg/kg/hour, from about 200 mg/kg/hour to about 250 mg/kg/hour, from about 250 mg/kg/hour to about 300 mg/kg/hour, from about 300 mg/kg/hour to about 350 mg/kg/hour, from about 350 mg/kg/hour to about 400 mg/kg/hour, from about 400 mg/kg/hour to about 450 mg/kg/hour, from about 450 mg/kg/hour to about 500 mg/kg/hour, from about 500 mg/kg/hour to about 550 mg/kg/hour, from about 550 mg/kg/hour to about 600 mg/kg/hour, from about 600 mg/kg/hour to about 650 mg/kg/hour, from about 650 mg/kg/hour to about 700 mg/kg/hour, from about 700 mg/kg/hour to about 750 mg/kg/hour, from about 750 mg/kg/hour to about 800 mg/kg/hour, from about 800 mg/kg/hour to about 850 mg/kg/hour, from about 850 mg/kg/hour to about 900 mg/kg/hour, from about 900 mg/kg/hour to about 950 mg/kg/hour, from about 950 mg/kg/hour to about 1000 mg/kg/hour, including any combination of endpoints thereof. Additionally, the method of internally administering the doses of the composition may vary from dose to dose (e.g. injection vs. inhalant).

The compositions of the present disclosure are illustrated by the following non-limiting examples, it being understood that these examples are intended to be illustrative only and that the present application is not intended to be limited to the materials, conditions, process parameters and the like recited herein. All proportions are by weight unless otherwise indicated.

### EXAMPLES

Two studies were performed using a composition of the present disclosure containing 1 wt % N-chloro-4-carboxybenzenesulfonamide (BENZ), 0.065 wt % sodium bicarbonate, and the balance water (“Composition X”).

### Example 1

Adult mice (n=8) were anesthetized, partially shaved, and the exposed skin was sterilized by chlorohexidine and then using a 70% alcohol gauze pad. Approximately 1-inch incisions were made from the lower neck to the upper back exposing the facial layer of tissue above the muscle. A 1 cm pre-soaked silk suture was implanted subcutaneously. Prior to implantation, the suture was soaked in a solution having a concentration of 10⁸ CFU/ml of methicillin-resistant Staphylococcus aureus (MRSA) for 1 hour. The incisions were closed using surgical glue.

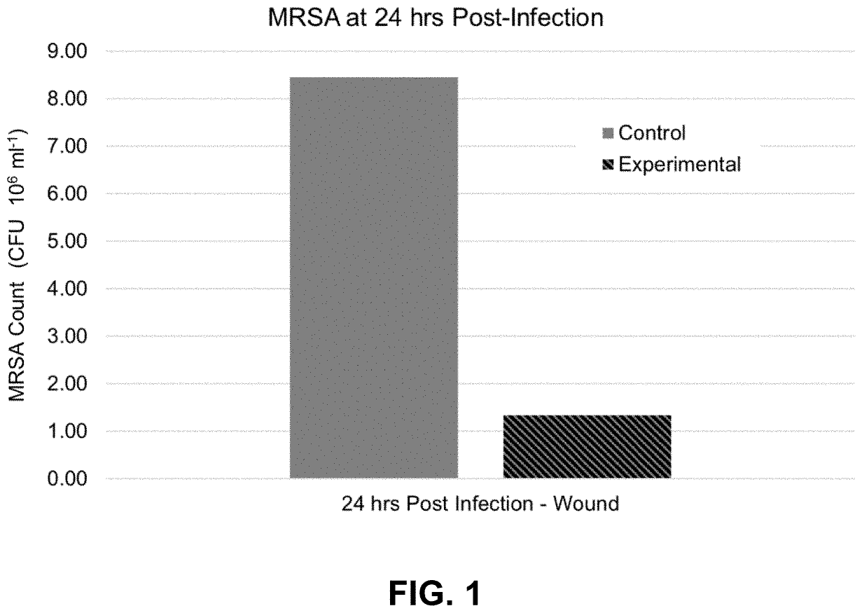

After 1 hour, half of the mice (n=4) were given 1 milligram of Composition X via intraperitoneal injection. A second dose of 1 milligram of the same inventive composition was given 18 hours after the first injection. A total of 2,000 micrograms of the halo active aromatic sulfonamide compound was administered. After 24 hours, the mice were euthanized and a tissue sample from the incision wound was harvested for testing. The bacterial load quantification results for the harvested tissue samples are shown below in Table A and illustrated in FIG. 1:

TABLE A
MRSA Infection at 24 hours post infection at the wound site

| | Subject | Infection Concentration (CFU/ml) |
|---|---|---|
| Untreated | A1 | 9.00E+05 |
| | A2 | 8.30E+06 |
| | A3 | 6.20E+05 |
| | A4 | 2.40E+07 |
| Untreated Average: | | 8.46E+06 |
| Treated w/Composition X | B1 | 2.30E+06 |
| | B2 | 1.20E+06 |
| | B3 | 2.10E+04 |
| | B4 | 1.80E+06 |

| | |
|---|---|
| Treated Average: | 1.33E+06 |

As shown in Table A and illustrated in FIG. 1, at 24 hours following infection with MRSA, the untreated subjects exhibited a significantly higher presence of infectious bacteria at the wound site than the subjects treated with Composition X. In particular, there was an 84.27% reduction in the infectious cell count between the untreated and the treated subjects.

### Example 2

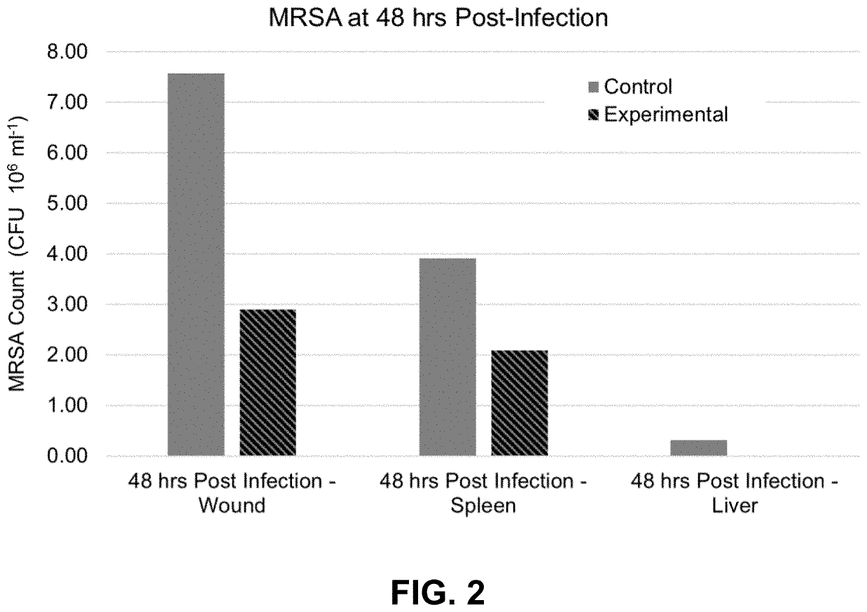

The same procedure and Composition X were used for Example 2 as Example 1, except that each subject was given a total of 750 micrograms of the halo active aromatic sulfonamide compound (i.e. via Composition X). After 48 hours, the mice were euthanized, and tissue samples from the wound site, the liver, and the spleen of each specimen were harvested for testing. The bacterial load quantification results for the harvested tissue samples are shown below in Tables B, C, and D, and illustrated in FIG. 2:

TABLE B
MRSA Infection at 48 hours post infection at the wound site

| | Subject | Infection Concentration (CFU/ml) |
|---|---|---|
| Untreated | A1 | 1.10E+07 |
| | A2 | 6.70E+06 |
| | A3 | 4.60E+06 |
| | A4 | 8.00E+06 |
| Untreated Average: | | 7.58E+06 |
| Treated w/Composition X | B1 | 8.00E+06 |
| | B2 | 2.60E+06 |
| | B3 | 5.00E+05 |
| | B4 | 5.00E+05 |

| | |
|---|---|
| Treated Average: | 2.90E+06 |

As seen above, observed was a 61.72% reduction in the presence of infectious bacteria at the wound site in the subjects treated with Composition X than when compared with the untreated subjects.

TABLE C
MRSA Infection at 48 hours post infection in the spleen

| | Subject | Infection Concentration (CFU/ml) |
|---|---|---|
| Untreated | A1 | 1.33E+05 |
| | A2 | 8.62E+03 |
| | A3 | 1.18E+04 |
| | A4 | 2.98E+03 |
| Untreated Average: | | 3.91E+04 |
| Treated w/Composition X | B1 | 2.73E+03 |
| | B2 | 8.05E+04 |
| | B3 | 0.00E+00 |
| | B4 | 0.00E+00 |

| | |
|---|---|
| Treated Average: | 2.08E+04 |

TABLE D
MRSA Infection at 48 hours post infection in the liver

| | Subject | Infection Concentration (CFU/ml) |
|---|---|---|
| Untreated | A1 | 1.86E+03 |
| | A2 | 0.00E+00 |
| | A3 | 1.05E+04 |
| | A4 | 2.81E+02 |
| Untreated Average: | | 3.16E+03 |
| Treated w/Composition X | B1 | 0.00E+00 |
| | B2 | 0.00E+00 |
| | B3 | 0.00E+00 |
| | B4 | 0.00E+00 |

| | |
|---|---|
| Treated Average: | 0.00E+00 |

As seen above, observed was a 46.78% and a 100% reduction in the presence of infectious bacteria in the spleen and liver, respectively, in the subjects treated with Composition X when compared with the untreated subjects.

### Example 3

Adult mice (n=16) received a ˜20% flame burn injury, followed by resuscitation with two doses of 0.5 mL of saline and 25 mg of tramadol for analgesia. After 5 minutes, they were infected with P. aeruginosa subcutaneously under the burn wound. 20 minutes post injury, five of the animals received 1 mg of BENZ subcutaneously in 0.25 ml saline, followed by six doses of 0.5 mg of BENZ subcutaneously also in 0.25 ml saline, approximately 8-12 hours apart. The animals were observed several times daily for seven days.

Of the 11 control animals that did not receive BENZ, 4 of 11 survived. Of the five animals that were treated with the drug, all five survived. The Fisher exact test statistic value was 0.0337, thus the result was significant at p<0.05.

The present disclosure has been described with reference to exemplary embodiments. Modifications and alterations will occur to others upon reading and understanding the preceding detailed description. It is intended that the present disclosure be construed as including all such modifications and alterations insofar as they come within the scope of the appended claims or the equivalents thereof.

1. A method for treating a patient with a composition, wherein the patient is suffering from an internal disease or is at risk of developing an internal disease, the method comprising the steps of:
determining a dosage of the composition; and
administering the dosage of the composition to the patient;
wherein the composition comprises a halo active aromatic sulfonamide compound of Formula (I): 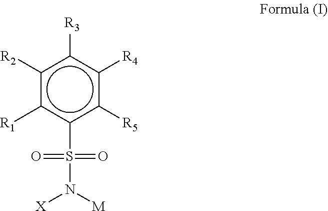
wherein R₁, R₂, R₃, R₄, and R₅ are independently selected from hydrogen, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic;
R′ is hydrogen, an alkali metal, an alkaline earth metal, substituted C₁-C₁₂ alkyl, or unsubstituted C₁-C₁₂ alkyl; and
R″ is hydrogen or substituted or unsubstituted C₁-C₁₂ alkyl, where the two R″ groups in CON(R″)₂ and N(R″)₂ may be independently selected;
X is halogen; and
M is an alkali or alkaline earth metal.

2. The method of claim 1, wherein the disease is at least one of a viral infection, a bacterial infection, and a fungal infection.

3. The method of claim 1, wherein the composition is internally administered to the patient via at least one of: oral administration, pulmonary administration, subcutaneous administration, sublingual administration, ocular administration, otic administration, intravenous administration, inter-dermal administration, epidural administration, intraperitoneal administration, intramuscular administration, intrathecally, nasally, opthalmically, rectally, topically, enterally, transdermally, buccally, vaginally, or intraurethrally.

4. The method of claim 1, further comprising the steps of:
after determining a dosage of the composition, providing an amount of the composition to a delivery device;
wherein the dosage of the composition is administered to the patient by contacting the delivery device with an internal portion of the patient, the delivery device being at least one of: a medical device; a medical instrument; a medical implant; and a prosthetic; and
wherein the amount of the composition provided to the delivery device is sufficient to deliver the determined dosage of the composition.

5. The method of claim 1, wherein the patient is a human or an animal.

6. The method of claim 1, wherein the dosage of the composition is internally administered to the patient in two or more doses over a treatment period, wherein the treatment period is from about one hour to about three days.

7. The method of claim 1, wherein the composition is internally administered to the patient to target a specific organ, wherein the specific organ includes at least one of: liver; spleen; colon; intestine; a muscle; kidney; heart; pancreas; gallbladder; lung; and brain.

8. The method of claim 1, wherein the infection includes at least one of: human immunodeficiency virus (HIV), hepatitis; Pseudomonas; methicillin-resistant Staphylococcus aureus (MSRA); vancomycin-resistant enterococci (VRE); Streptococcus; Staphylococcus; Escherichia coli (E. coli); Klebsiella; Salmonella; Clostridium difficile (C. diff.), Candida; Acinetobacter baumannii; and influenza.

9. The method of claim 1, wherein the infection includes a drug-resistant infection.

10. The method of claim 1, wherein the disease is at least one of a blood disorder, a parasite, and a type of cancer.

11. The method of claim 1, wherein the composition is in the form of an aerosol, a capsule, an emulsion, a film, a gel, granules, a gum, an injection, a lozenge, a paste, pellets, a pill, a powder, a solution, a spray, a suppository, a suspension, a tablet, or a tape.

12. A composition for internal administration in a patient and effective for the treatment or prevention of an infection in the patient, the composition comprising:
a halo active aromatic sulfonamide compound of Formula (I): 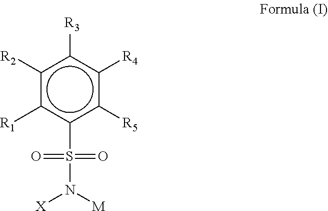
wherein R₁, R₂, R₃, R₄, and R₅ are independently selected from hydrogen, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic;
R′ is hydrogen, an alkali metal, an alkaline earth metal, substituted C₁-C₁₂ alkyl, or unsubstituted C₁-C₁₂ alkyl; and
R″ is hydrogen or substituted or unsubstituted C₁-C₁₂ alkyl, where the two R″ groups in CON(R″)₂ and N(R″)₂ may be independently selected;
X is halogen; and
M is an alkali or alkaline earth metal.

13. The composition of claim 12, wherein at least one of R₁, R₂, R₃, R₄, and R₅ is not hydrogen.

14. The composition of claim 12, wherein the halo active aromatic sulfonamide compound has the structure of Formula (II): 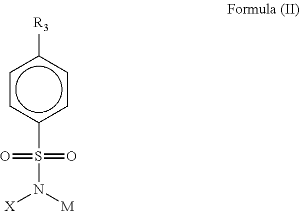
wherein R₃ is COOR′;
R′ is hydrogen, an alkali metal, an alkaline earth metal, substituted C₁-C₁₂ alkyl, unsubstituted C₁-C₁₂ alkyl, substituted aromatic, or unsubstituted aromatic;
X is halogen; and
M is an alkali or alkaline earth metal.

15. The composition of claim 14, wherein the halo active aromatic sulfonamide compound is N-chloro-4-carboxybenzenesulfonamide.

16. The composition of claim 12, further comprising water.

17. The composition of claim 12, further comprising a buffering agent.

18. The composition of claim 12, further comprising a surfactant.

19. The composition of claim 12, wherein the composition comprises from about 0.0001 wt % to 100 wt % of the halo active aromatic sulfonamide compound.

20. A delivery device comprising the composition of claim 12, wherein the delivery device is a medical device; a medical instrument; a medical implant; or a medical prosthetic.
